(19) United States
(12) Patent Application Publication
(10) Pub. No.: US 2005/0112758 A1
(43) Pub. Date: May 26, 2005

(54) Method and apparatus for the storage of a tissue specimen

(76) Inventors: Maeghan Archambault, Vernon, CT (US); Mark Broyles, Plainville, CT (US)

Correspondence Address: McCarter & English, LLP;CityPlace I, 185 Asylum Street, Hartford, CT, 06103-3495, US

(21) Appl. No.: 10/722,947

(22) Filed: Nov. 26, 2003

(51) Int. Cl. C12M 1/00

(52) U.S. Cl. 435/307.1; 435/40.52

(57) ABSTRACT

A device is provided for transporting a tissue specimen without the risk of exposing health-care workers to potentially hazardous tissue fluids. The device includes a container, a flexible portion, a first support member received in the container, a second support member extending from the fist support member, a first locating indicia on a first side of the first supporting member, and a second substantially radiopaque locating indicia on a second side of the first support member, and an indicating member movably mounted on the first side of the first support member. The first support member has a first locating indicia on a first surface and a second locating indicia on a second surface, which generates a radiographic image when exposed to x-rays. The first and second indicia are substantially in registration such that when a tissue specimen is positioned on the first locating indicia and is then exposed to x-rays, a radiographic image of the specimen superimposed on the image of the second locating indicia is produced. Since both locating indicia are in registration, any tissue abnormality within the specimen can be precisely located with respect to both indicia. The device further includes a second support member, limiting contact of the container walls with the tissue specimen, and biasing the flexible portion away from the first support surface containing the tissue specimen. The device further includes an at least partially radiopaque indicating member for indicating the position of the tissue specimen on the radiographic image. A method for using the device to generate radiographic images of a tissue specimen is also disclosed.

### FIELD OF THE INVENTION

The present invention relates generally to a method and apparatus for use in the storing and/or manipulation of tissue specimens, such as for storing and/or manipulating tissue specimens in the course of producing image radiographs of tissue specimens.

### BACKGROUND OF INVENTION

Diagnostic radiology procedures often show a tissue abnormality which can only be identified as either malignant or benign by surgical biopsy and subsequent microscopic study. This is most often the case with mammography, the examination of the breast with radiography, where a suspected soft tissue tumor or an abnormal calcification seen on a mammogram frequently is not palpable due to its small size.

There are roughly 750,000-1,000,000 surgical breast biopsies performed in the U.S. each year. Of these, roughly 55% are needle localized. In needle localization a hook located on the end of a wire is placed directly into the area of concern, and once the wire is in place, the patient is taken to the operating room (OR). The surgeon, guided by an implanted guide wire, cuts around the hook to remove a block of tissue thought to contain the abnormality.

Once out of the patient, the specimen is transported to radiology for imaging. A radiologist then examines the tissue block by specimen radiography to determine if it does in fact contain the suspicious tissue. If the radiologist does identify the same abnormal tissue features seen during the initial radiological examination, the surgical portion of the procedure is concluded, and the surgeon begins closing the suture. The specimen is then transported to pathology, usually with a copy of the specimen radiograph.

The abnormal tissue, which is frequently less than a centimeter in diameter, must then be localized within the larger tissue block, which usually ranges between 6 to 10 cm in diameter. Typically, the pathologist responsible for performing the microscopic examination of the abnormality cannot accurately identify it either by feel or by gross tissue sectioning. Thus, the radiologist must assist the pathologist by precisely describing the area within the tissue block where the abnormality is located. Once the abnormality has been accurately localized, the pathologist excises a segment measuring 10 to 15 mm in diameter for fixation, sectioning, staining and microscopic examination.

For years, the purpose of the specimen radiograph was simply to verify that the area of interest was removed from the patient. Recently though, radiologists have been looking at the margins of the breast specimens on film. A margin is the distance between the area of interest and the outermost edge of the specimen. There are an infinite number of margins as this line between the area of interest and the outermost edge can be drawn at any point on the specimen. The goal of the surgeon is to remove healthy tissue on all sides of the suspicious area. Many attempt a 1 cm margin but the optimal margin size can vary by surgeon.

Only a pathologist can definitively say whether enough of a margin was obtained, but the specimen radiograph can serve as a first check for anything that is obviously too close to the edge of the specimen. The benefit for the patient is that if a close margin is caught in radiology, the radiologist can tell the surgeon while the patent is still in the OR. If a close margin is not seen until pathology, the patient will have to come back for a second surgery to excise more tissue.

The challenge in radiology is viewing the margins of a three dimensional specimen on a two dimensional image. Traditional one-view specimen imaging only assesses superior, inferior, medial, and lateral sides. The anterior and posterior aspects cannot be evaluated because depth cannot be assessed on a two dimensional image. A recent improvement in the radiological techniques employed by radiologists includes the taking of orthogonal images of a biopsied tissue specimen. Orthogonal imaging is the practice of taking multiple images of the specimen, 90 degrees apart. A recent survey indicated that only 10% of hospitals that do surgical breast biopsies are performing orthogonal imaging. When the specimen is turned 90 degrees and reimaged, what used to be anterior and posterior now becomes medial and lateral. The new position of these margins allows for radiographic evaluation. The more margins a radiologist can see on film, the better chance he or she has of catching a close margin radiographically, while the patient is still in the OR. The addition of an orthogonal view could save a patient from a second surgery.

Unfortunately, radiologists, lab technicians, and other health-care workers find it difficult to handle and radiograph tissue specimens, and at the same time, prevent personal exposure to tissue fluids and equipment contamination. This is of particular concern in view of a recent program instituted by the Occupational Safety and Health Administration (OSHA) to enforce safety standards intended to limit occupational exposure to body tissues, blood, and other tissue fluids. Pursuant to these standards, every employer is required to anticipate and identify any such exposure in the workplace and provide protective equipment, an exposure control plan, appropriate safety devices, and an employee educational program. Further, all body fluids and tissues from all patients must be treated as potentially lethal. At a minimum these so-called “universal precautions” will require gloves, gowns, masks, face shields, and safe needle and tissue fluid disposal when conducting tissue specimen radiography.

Thus, it would be desirable to provide a method and an apparatus wherein a tissue specimen can be transported, manipulated, and examined radiographically or by ultrasound without the risk of exposing health-care workers to potentially hazardous tissue fluids or contaminating equipment with such fluids.

### SUMMARY OF THE INVENTION

According to one aspect of the present invention, a device includes a transparent, sealable, liquid impervious container for receiving a tissue specimen. The transparent, sealable, liquid impervious container also includes on a first side a sealable opening, and on a second side an at least one flexible portion. In a currently preferred embodiment of the present invention, the flexible portion is located on a side opposite the sealable side. Positioned inside the container is a first support member, for example a tray, which has an alpha-numeric marked indicia. In a preferred embodiment, the indicia is a grid formed by a series of intersecting lines. The intersecting lines form individual sectors which allow a health-care worker to see, by eye, the relative position of the tissue specimen and/or any abnormality. Various examples of embodiments of grid lines are given in U.S. Pat. No. 5,383,234, which is herein incorporated by reference into the present application in its entirety. The first support member may contain a first or visible indicia on a first surface and a substantially radiopaque indicia on the opposite, or second surface. In another embodiment, the visible indicia also may be the radiopaque indicia, or the first support member may contain some combination of the two. The visible and radiopaque locating indicia are in registration with one another; thus, when the tissue specimen is placed on the first support member within the boundaries of the visible indicia and exposed to x-rays, any abnormality within the specimen appears at precisely the same location on both indicia. Accordingly, the location of the abnormality within the specimen can be accurately determined.

The first support member is formed from a substantially fluid impervious material which prevents tissue fluids from completely penetrating through from the first side of the first support member to its opposite side. Accordingly, any tissue fluid contacting the first side of the first support member cannot completely penetrate it and interfere with the second locating indicia. In a preferred embodiment of the invention, the first support member is substantially a tray formed from fluid impervious cardboard. The preferred embodiment also includes a thin, clay coating on the first side of the first support member which substantially immobilizes the tissue specimen to the first side of the first support member and prevents movement within the first locating indicia.

The device further includes a second support member extending from the first support member for limiting contact of the container walls with the tissue specimen. Unless otherwise defined herein, the phrase “extending from,” in reference to the second support member, is meant to include, but is not limited to, embodiments where the second support member is glued, pinned, clamped, or formed integrally with the first support member, and includes embodiments where the second support member is substantially similar or different in height, width, or length, as the first support member. As such, an air pocket is created between the container walls, and the tissue specimen sitting on the tray surface. One advantage of this feature is that unintentional contact of the tissue specimen with the container walls is avoided, thereby allowing for convenient manipulation of the specimen by a health-care worker in order to take orthogonal images of the specimen. The second support member also is adapted to act as a spring serving to bias the flexible portion of the container away from the radiographic field of the first locating indicia. One advantage of this feature is that the radiographic image is not obscured by the flexible portion.

The device further includes an at least partially radiopaque sequence indicating member that is movably mounted on the first support member for indicating on a radiographic image the position of the tissue specimen relative to the locating indicia on the first support member. In a preferred embodiment, the indicating member is made of plastic, for example polystyrene. At one end of the sequence image-indicator is a fastener. The fastener may be, for example, a hole through which a screw (plastic) or a pin is used to mate the sequence image-indicator to a thread or hole in the grid, or a pin to mate to the hole in the grid. In another embodiment, the indicating member is integral with the first support member.

In a preferred embodiment, the indicating member is disposed to a first position indicative of a first radiographic image of a tissue specimen located on the support in a first position, and then disposed to at least one second position indicative of at least one second radiographic image of the tissue specimen located on the support in at least one second position. The indicating member may be held in position, for example, a first position, through the use of a protrusion (a short pin or hemisphere, for example) receivable within corresponding recesses formed in the first support member to keep the indicating member in any of the positions engraved on the first support member.

The device further includes means for securing the tissue specimen to the first side of the tray in a fixed position. In a preferred embodiment, the tissue is held in place by means of a radiolucent, clay coating on the fist locating indicia on the first side of the first supporting member once the specimen is compressed. While this is the preferred means for maintaining the specimen in a fixed position on the first support member, other suitable means also may be employed. For example, an absorbent material can be combined with the cardboard from which the first support member is formed. Preferably, only enough absorbent material is added to permit tissue fluid absorption at the surface of the first support member when the tissue specimen is placed thereon. It has been found that this limited absorption is sufficient to maintain the specimen in a fixed position with respect to the first support member. In another embodiment, the first support member is coated with a radiolucent adhesive that bonds the specimen to the first support member. Clamping means also may be employed as long as such means do not interfere with the radiographic image of the specimen and the second locating indicia.

Preferably, the container is formed from a flexible material which permits manipulation of the tissue specimen from outside of the container. Thus, radiologists and other health-care workers can position the tissue specimen on the first support member without exposure to tissue fluids. The container is also preferably formed from a material which permits the uninterrupted transmission of sound waves so that ultrasound images of the specimen also can be generated.

In another aspect, the invention relates to a method for transporting and radiographically examining a tissue specimen using orthogonal imaging techniques without the risk of exposing health-care workers to tissue fluids. A radiographic device is provided which includes a container, a flexible portion, a first support member received in the container, a second support member extending from the fist support member, a first locating indicia on a first side of the first supporting member, a second substantially radiopaque locating indicia on a second side of the first support member, and an indicating member movably mounted on the first side of the first support member. The tissue specimen is positioned on the first support member, for example a tray, in a fixed position, and then sealed within the container. The tissue specimen is then exposed to x-ray radiation while the specimen is fixed to the tray and sealed in the container with the indicating member set to a first position to generate an x-ray image of the specimen superimposed on the second locating indicia, for example, an alpha-numerically marked grid. The health-care worker then manually inserts a hand or fingers, or both, into the flexible portion to grasp and/or rotate the tissue specimen, for example, rotating the specimen approximately 90 degrees. Again, the tissue specimen is exposed to x-ray radiation while the specimen is fixed to the tray and sealed in the container with the indicator member set to a second position to generate an x-ray image of the specimen superimposed on the second locating indicia. It should be understood by one of ordinary skill in the art that this method could include an embodiment with an indicator means having more than two positions, and the indicating member disposed to a different position for each new image. In addition, rather than generating x-ray images, the method may involve generating ultrasound, MRI or other diagnostic image.

Accordingly, one advantage of the present invention is that it not only provides a health-care worker the ability to transport a tissue specimen in a flexible, sealable, fluid-impervious container for radiographic imaging, but the extension portion allows the health-care worker to manipulate the tissue specimen easily, and free from risk of contamination. Also, the addition of the second support member biases the flexible portion away from the tissue specimen so that the radiographic image is not obscured. The addition of the indicator member also provides the worker with a means to avoid confusion over the orientation of the tissue specimen represented on the radiograph. Other advantages of the present invention will become more readily apparent in view of the following detailed description of the currently preferred embodiments and accompanying drawings.

### BRIEF DESCRIPTION OF THE DRAWINGS

FIG. 1 is a perspective view of a preferred embodiment of a device for storage of a tissue specimen and embodying the present invention.

FIG. 2 is a schematic view of the container of the device of FIG. 1 with the flexible portion fully protracted out of the container.

FIG. 3 is a schematic view of the container of the device of FIG. 1 with the flexible portion inverted within the container.

FIG. 4 is a perspective view of the first surface of the first support member of the device of FIG. 1, including the first locating indicia with alpha-numeric labeling, the second support members, and the indicating member.

FIG. 5 is a perspective view of the second surface of the first support member of the device of FIG. 1, including the second locating indicia.

FIG. 6 is a perspective view of the device of FIG. 1 demonstrating the use of the flexible portion and the biasing of the second support member.

### DETAILED DESCRIPTION OF THE INVENTION

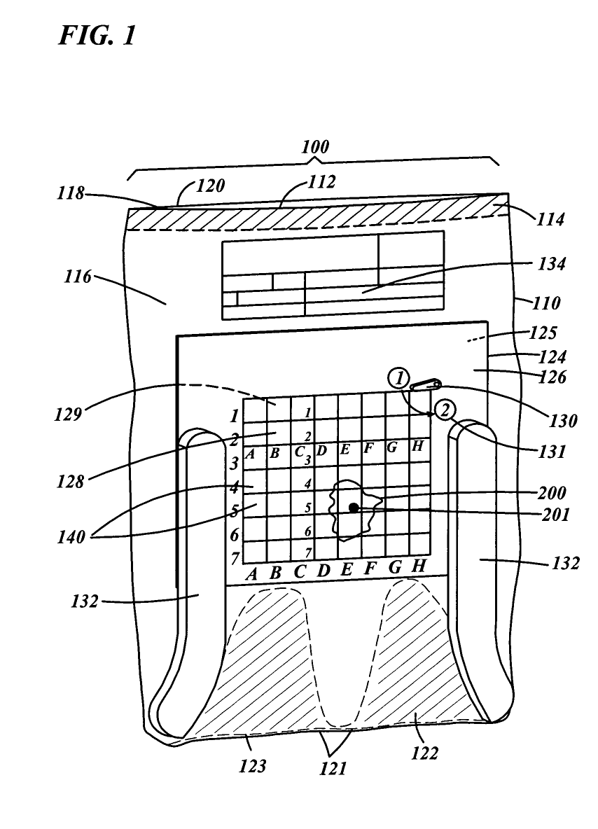

FIGS. 1-6 illustrate a first embodiment of a device for storing and/or for taking orthogonal images for radiographic examination of a tissue specimen. In FIG. 1, the device 100 comprises a flexible, transparent, sealable, liquid impervious bag 110 for receiving a tissue specimen 200 having an abnormality contained therein. In the preferred embodiment the bag has an opening 112 provided with a sealable adhesive strip 114. However, as should be obvious to one of ordinary skill in the art, opening 112 may be sealed by any suitable means for providing a fluid-tight seal to the opening 112 of the bag. In another embodiment the opening 112 is substantially resealable and liquid impervious, for example through the use of a Ziploc®-type device. The opening 112 provides access to a chamber or pocket 116 defined by walls 118 and 120 for receiving the specimen. In general, the pocket 116 is large enough to accommodate multiple tissue specimens from the same patient, each specimen measuring up to 12 or more centimeters in diameter.

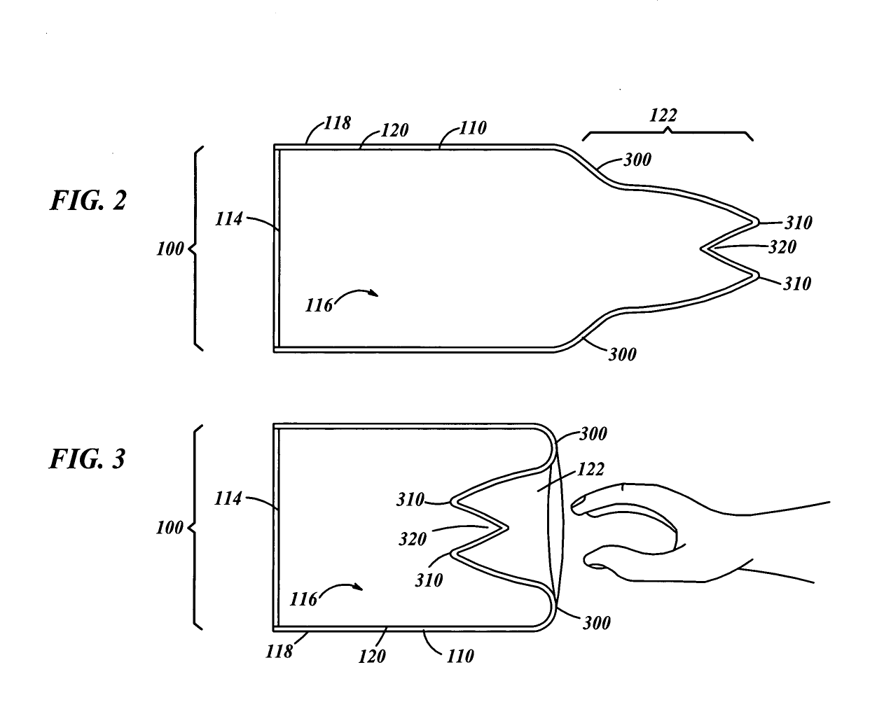

The bag 100 also comprises a flexible, transparent, sealed, liquid impervious extension portion 122 (i.e., a flexible portion). The flexible portion 122 is contained within the bag 110 on a side other than the side containing sealable opening 112. The flexible portion 122 is accessed, by tearing a perforation 123 extending along the adjacent edge of the bag 110 to thereby separate the opposing edges and form an access opening 121 there between. Alternatively, the opposing sedges of the opening 121 may be releasably secured by an adhesive, glue or other releasable securing mechanism that is currently or later becomes known, or may not include any such releasable securing mechanism at all. The flexible portion 122 then forms a fluid-impervious sheath allowing the health-care worker to insert an operating member, such as a hand or a finger, and to grasp rotate or otherwise manipulate the tissue specimen from outside of the container, free from risk of contact with the specimen. FIG. 2 illustrates the general structure of the bag 110 and flexible portion 122 prior to folding the flexible portion 122 into the interior of the body. As can be seen, in the unfolded state, the flexible portion 122 defines a protuberance extending laterally from the main portion of the bag 110. In the present embodiment, the bag 110 is about 9 inches wide and about 12 inches long, while the flexible portion 122 is delimited by a slight taper 300 and further extends along its length for about two-thirds the total length of the body portion 116. At the distal most point 310 of the flexible portion 122 a cleft is formed 320 such that when the flexible portion is inverted into the body of the bag 116, the cleft 320 and distal points 310 form sheaths, flexibly, and sealingly adapted for the manipulation of the tissue specimen 200 (see also FIG. 3). It should be obvious to one of ordinary skill in the art that the presently disclosed shapes, and dimensions of the bag 110 and flexible portion 122 are given by way of example only, and may exist in any of various shapes, widths, lengths, and sizes.

While the flexible portion 122 illustrated in FIG. 1 contains a two-part sheath, it will be understood by one of ordinary skill in the art that the flexible portion may exist in any one of various other embodiments. Another example includes a flexible portion formed simply from an excess of bag material which can be inverted into the body of the bag 116 and used to manipulate tissue specimen 200. Further examples include a flexible portion with a plurality of sheaths. For example, extension portion 122 could contain a plurality of sheaths substantially shaped like at least one glove inverted into the body of the container.

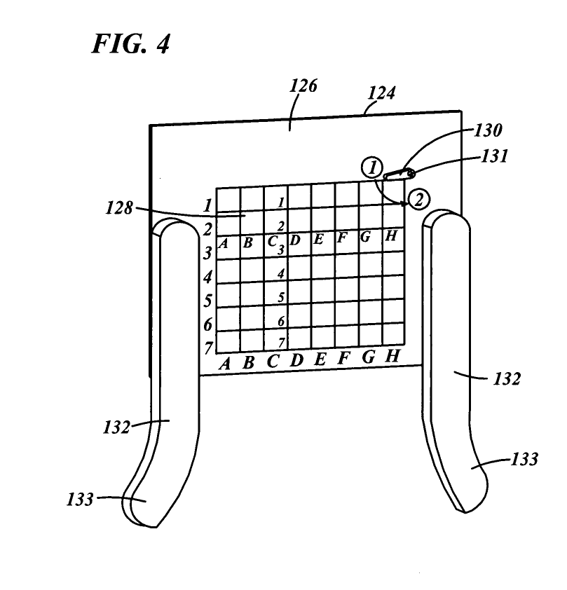

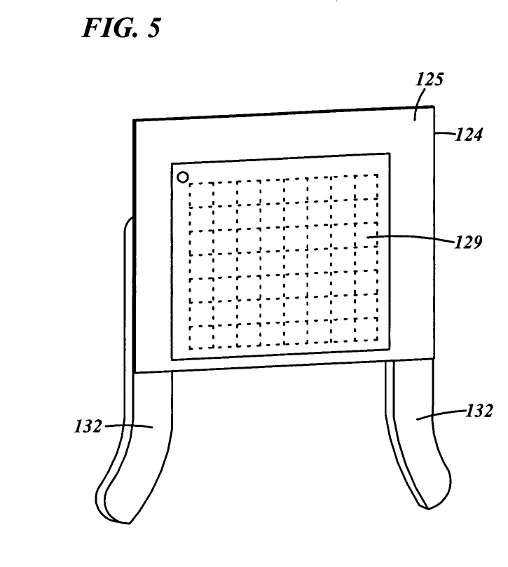

FIG. 4 illustrates the first support member. In the preferred embodiment the first support member is a tray 124 that supports the tissue specimen 200. The tray has a first side or surface 126 with a first locating indicia, which in the currently preferred embodiment is a grid, with alpha-numeric markings 128 printed thereon. FIG. 5 shows the second surface or side 125 of the first support member 124, which can contain a radiographic second locating indicia with alpha-numeric markings 129, which in the currently preferred embodiment is a grid, in registration with the first locating grid 128 on the first side of the tray. The grid 128 is formed from a low density material, and the grid lines and alpha-numeric designations comprising grid 129 generate a radiographic image when exposed to x-rays. Preferably, grid 128 is made from a plastic, and the grid lines and alpha-numeric designations are grooves formed in the plastic. In a preferred embodiment, air within the groove portions provides them with a greater radiolucency relative to that of the adjacent plastic portions. To maintain the correct radiopacity/radiolucency of the grooves, they must be kept free of fluid. This is accomplished by directly attaching grid 128 to side 126 of the tray to seal the grooves against the entry of fluid. Like the bag 110, the tray 124 is large enough to accommodate multiple specimens from the same patient.

FIG. 4 also illustrates a radiopaque image indicating member 130. The indicating member 130 is movably mounted and disposed to one of at least two positions to clearly indicate on a radiograph the orientation of the tissue specimen relative to the first surface of the first support member. In the illustrating embodiment, the indicating member 130 is in the form of a substantially radiopaque pointer that is pivotably mounted at one end by a pin 131 to the support surface 126. In accordance with a method in the present invention, the indicating member 130 is set to a first position (#1 in FIG. 4) prior to generating a first radiological image. The health-care worker can then manipulate the tissue specimen, for example, by rotating the specimen 90 degrees for an orthogonal image, and then disposes the indicating member 130 to a second position (#2 in FIG. 4) so as to indicate, on the radiograph, that the image is in an orthogonal position, and allow the radiologist to be able to distinguish the orthogonal image from the original image. In a preferred embodiment, the indicating member is a lever 130 which is pivotably movable between a first position (#1) and a second position (#2). It should be understood by a person of ordinary skill in the art that the radiopaque indicating member may assume any number of different embodiments, such as, for example, but not limited to, a circular dial or a linear dial, each containing at least two positions, respectively.

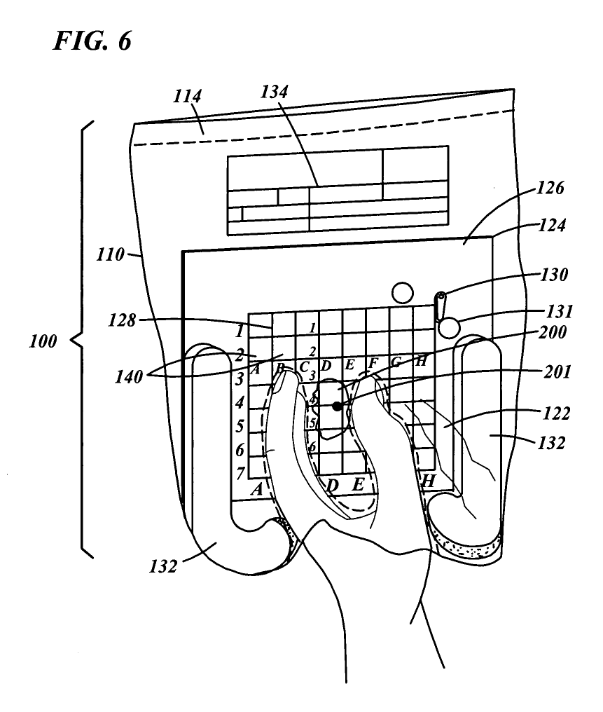

The first support member 124 of FIGS. 4 and 5 also comprises at least one second support member 132, and preferably two as shown. The second support members 132 limit contact of the walls of the container 110 to the tissue specimen by forming an air pocket between the specimen and the container walls 118 and 120. The presence of this air pocket makes it more convenient for the health-care worker to manipulate the tissue specimen for orthogonal imaging, and prevents radiographic anomalies due to fluid build up at the tissue specimen/container wall interface. In a preferred embodiment, the second support member is formed by two substantially elongated block-type low density structures 132, for example foam, placed at opposite sides of the tray 124. More specifically, each second support member 132 includes a biasing portion 133 that extends outwardly beyond the edge of the tray 124. The two biasing portions 133 also extend laterally outwardly in opposite directions relative to each other into engagement with the adjacent side walls of the bag 110 (FIG. 1). As described further below, when the flexible portion 122 is moved inwardly by a user to grasp and/or manipulate a specimen and is then released, the biasing portions 133 bias the bag and flexible portion 122 thereof away from the specimen and into the imaging position shown, for example, in FIG. 1. The second support member 132 also functions substantially as a spring to bias the flexible portion 122 away from the first support member to avoid obscuring the radiographic image (FIG. 6). It should be understood by a person of ordinary skill in the art that the size, shape, and material of the second support member 132 may assume any number of different embodiments.

As those skilled in the art well-know, compression of the specimen is a very important aspect of specimen radiography. Thus, after the surgeon removes the tissue specimen from the patient, and it is placed on the first surface 126 of the first support member 128, see FIG. 1, the pocket 112 is sealed. The specimen is then transported to radiology for examination. Compression tends to displace air from the tissue and produces a more uniform tissue thickness and uniform fine image density. In actual practice, the bag 110 containing the tissue specimen is placed on a standard mammography x-ray table or tray and an associated paddle is lowered to compress the tissue. The degree of compression can be controlled by the operator, and mild to moderate compression typically is necessary to obtain the desired uniform thickness.

Those skilled in the art also recognize that some tissue abnormalities, particularly breast abnormalities, can only or primarily be visualized by ultrasound. Since such abnormalities may also not be palpable, they must be localized by ultrasound prior to surgery. Further, after surgical biopsy, the only way to be sure that such an abnormality is present in the removed tissue specimen is to examine the specimen with ultrasound.

If desired, and to meet these objectives, the bag 110 may be made from a flexible material which permits mild to moderate compression of the specimen while it is sealed within the bag. Moreover, the material forming the bag may also provide for the uninterrupted transmission of sound waves so that the specimen may be subjected to adjunctive ultrasound imaging. In the illustrated embodiment, the bag 110 is made from plastic, although any other flexible material meeting these criteria would, of course, be acceptable. The bag's flexibility also permits the radiologist or other health care workers to further position the specimen with respect to grid 128 without direct contact with the specimen. Thus, the specimen is transported and manipulated for radiographic examination without the risk of exposing health-care workers to possible hazardous tissue fluids and without the risk of equipment contamination.

Referring again to the first support member 124 of FIGS. 4 and 5 in more detail, in the preferred embodiment the first support member is formed from a substantially fluid impervious cardboard which is free from radiopaque artifacts and patterns. The cardboard includes a quantity of absorptive material sufficient to permit limited absorption of tissue fluid at the surface of the first support member. The first support member is constructed in this manner because tissue fluid absorbed on side 126 has proven effective in maintaining the specimen in a fixed position with respect to the first support member while the specimen is being transported, examined and sectioned. In the illustrated embodiment, the absorptive material is an absorptive paper combined with the cardboard. It has been found that Carolina Coated Blank paper available from Federal Paper Co. is particularly useful in this regard.

In this embodiment, it is desirable that fluid absorption be limited to the first surface of the first support member only, since any fluid penetrating entirely through to the second surface 125 of first support member 124 might enter the grooves forming radiographic second locating indicia 129. If this occurs, fluid entering the grooves might interfere with the radiographic shadow cast by the second locating indicia. Since the grooves formed in second locating indicia 129 are immediately adjacent side 126 of the first support member, the first support member seals at the grooves and prevents fluid from entering therein.

In a preferred embodiment of the device, bag 110 may further comprise an integral pocket defined by the container walls for storing requisition slips or other forms of documentation. The bag 110 is also provided with an integral patient identification label 134. Since the label 134 is integral with the bag, the chances of associating a particular tissue specimen with the wrong patient are greatly reduced. Further, the label may also include the locating indicia coordinates of the identified tissue abnormality, allowing direct communication of the coordinates to the pathologist without error, as will be explained in more detail below.

Turning now to a method of using the device 100 for specimen radiography, and with reference to FIGS. 1 and 6, it will be appreciated that since second locating indicia 129 generates a radiographic image when exposed to x-rays, an x-ray image of specimen 200, when positioned on first locating grid 128, will show the specimen superimposed on locating indicia 129 with the indicating member 130 set at a first position (#1). By referring to the radiographic image of the specimen superimposed on the second locating indicia 129, the pathologist may, for example, pinpoint the location of the abnormality within the specimen to within a few millimeters using the first locating indicia 128.

Thus, an abnormality, contained within the specimen, for example abnormality 201, can be identified and precisely located with respect to both indicia. Since the bag contains a flexible portion adaptable as a sheath, the specimen can be easily manipulated through the flexible portion and turned 90 degrees or otherwise as desired, and the indicating member set to a second position 131 for orthogonal imaging. Alternatively, the specimen may be conveniently palpated through the extension portion 122, which may allow the pathologist to either locate the abnormality by manual manipulation or locate an area within the specimen which may have been stained by localizing dye, such as methylene blue, as part of the surgical biopsy procedure.

Current mammography frequently includes radiographical magnification views of a breast for improved soft tissue detail and detailed examination of possible malignant calcitic deposits. The present invention permits similar corresponding specimen magnification views which may be compared to the original mammograms to ensure that the abnormal tissue is included within the excised specimen.

As will be appreciated by those skilled in the art, the density of the material from which radiographic second locating indicia 129 is formed and the construction of the grooves are preferably such that the second locating indicia generates a clear radiographic image which projects through the image of the specimen but at the same time does not obscure fine tissue detail. As noted above, the second locating indicia 129 is made of plastic, and the air within the grooves provides them with a greater radiolucency relative to that of the plastic. This is why it is desirable that fluid be kept from entering the grooves. In the case where the locating indicia are substantially a grid, it is also desirable that the size of the sectors 140, 140 defined by the grid lines be dimensioned to provide accurate localization of the abnormality without introducing confusing radiographic shadows. Generally, the sectors measure from about 10 to about 20 mm on a side. Finally, since the opening 112 is fluid-impervious and sealable, the first support member 124 can be placed within the bag, sealed, and the entire system sterilized to that it can be introduced without concern of contamination into the surgical field during the biopsy.

While preferred embodiments have been shown and described, various modifications and substitutions may be made without departing form the spirit and scope of the invention. For example, although the bag in the embodiments is transparent this is not absolutely required. The bag may have any of various degrees of transparency including for example, but not limited to, semi-transparent, translucent and/or a combination of transparent, semi-transparent, and translucent. Some portion of the bag may also be opaque. Similarly, although the length of the extension portion of the embodiment is roughly equal to two-thirds of the total length of the body portion of the bag, the extension portion may of any length or size that allows the worker to reach into the body of the bag and manipulate the tissue specimen. Also, although the extension portion of the embodiment is shown to be inverted to form a sheath substantially shaped like a glove, the sheath also may be adapted from an excess of bag material which forms a pocket when inverted into the body of the bag allowing a worker to manipulate the specimen, or the extension portion may be adapted to allow for the insertion of each finger of a worker's hand (or other respective members) into individual sheath portions to allow for increased dexterity. Similarly, the extension portion of the embodiment is located opposite the sealable side, however, the extension portion may be located on any side, for example, the top, left side or right side (relative to the sealable side), or multiple sides, or any combination of top, left side or right side with multiple extension portions. Additionally, the tray of the currently preferred embodiment includes a locating grid for determining the position of an abnormality within the tissue specimen, however, a grid is not absolutely required. The tray may contain any of various means for determining relative position, including, for example, concentric circles, parallel lines, or a meter bar in English or metric units alone or in combination with a grid and marked using any symbols, alpha-numeric representations, alone or in combination. Accordingly, it is to be understood that the present invention has been described by way of example and not by limitation.

1. A device for storing a tissue specimen comprising:
a sealable, substantially liquid impervious container; and
a first support member received within the container and having a first surface for supporting a tissue specimen;
wherein the container has a flexible portion that permits grasping and manipulation of the tissue specimen from outside the container.

2. A device as defined in claim 1, wherein the container is a flexible bag defining a sealable, substantially fluid-tight chamber, and the flexible portion is defined by a portion of the bag folded inwardly and extending into the chamber.

3. A device as defined claim 2, wherein the flexible bag is formed of plastic.

4. A device as defined in claim 1, wherein the first support member is formed substantially of a substantially rigid material.

5. A device as defined in claim 1, wherein the first support member contains a coating to prevent relative movement between the tissue specimen and first support member.

6. A device as defined in claim 1, wherein a first locating indicia is located on the first support member and visually represents the position of the tissue specimen on the first support member.

7. A device as defined in claim 6, wherein the first locating indicia is at least one of a line, a plurality of intersecting lines, a two-dimensional Cartesian plane, a grid pattern, a circle, a plurality of substantially concentric circles, and a combination of any of the foregoing.

8. A device as defined in claim 1, wherein the first support member is substantially liquid impervious.

9. A device as defined in claim 1, wherein the liquid impervious container permits the uninterrupted transmission of sound waves therethrough.

10. A device as defined in claim 1, wherein the flexible portion of the container includes at least one portion which folds inward and extends into the container.

11. A device as defined in claim 1, wherein the liquid impervious container contains a plurality of said flexible portions.

12. A device as defined in claim 1, wherein the liquid impervious container contains a plurality of said flexible portions, each with at least one portion which folds inward and extends into the container.

13. A device as defined in claim 1, wherein a second locating indicia is located on a second side of the first support member, and said second locating indicia is substantially radiopaque when viewed on a radiograph.

14. A device as defined in claim 13, wherein the second locating indicia is defined by a low density material forming grooves in the first support member.

15. A device as defined in claim 13, wherein the second locating indicia is at least one of a line, a plurality of intersecting lines, a two-dimensional Cartesian plane, a grid pattern, a circle, a plurality of substantially concentric circles, and a combination of any of the foregoing and is substantially in registration with the first locating indicia.

16. A device as defined in claim 15, wherein the low density material further includes alpha-numeric designations formed there.

17. A device as defined in claim 16, wherein the low density material is a plastic.

18. A device of claim 1, further comprising an indicating member that is at least partially radiopaque, and is movably mounted to the first support member, and wherein the indicating member is capable of being disposed to at least two positions indicative of at least a first radiographic image of a tissue specimen, and a second radiographic image of a tissue specimen.

19. A device as defined in claim 2, wherein the flexible portion defines a manually engageable portion that is locatable on substantially opposite sides of the tissue specimen relative to each other and is manually engageable by a user for grasping and manipulating the position of the tissue specimen on the support.

20. A device for storing a tissue specimen comprising:
a sealable, substantially liquid impervious container having a flexible portion;
a first support member received within the container having a first surface for supporting a tissue specimen; and
a second support member extending from said first support member for biasing at least a portion of said flexible portion away from the first support member.

21. A device as defined in claim 20, wherein the container is a flexible bag defining a sealable, substantially fluid-tight chamber.

22. A device as defined claim 21, wherein the flexible bag is formed of plastic.

23. A device as defined in claim 20, wherein the first support member is formed of a substantially rigid material.

24. A device as defined in claim 23, wherein the first support member contains a coating to prevent relative movement between the tissue specimen and first support member.

25. A device as defined in claim 20, wherein a first locating indicia is located on the first support member and visually represents the position of the tissue specimen on the first support member.

26. A device as defined in claim 25, wherein the first locating indicia is at least one of a line, a plurality of intersecting lines, a two-dimensional Cartesian plane, a grid pattern, a circle, a plurality of substantially concentric circles, and a combination of any of the foregoing.

27. A device as defined in claim 20, wherein the first support member is substantially liquid impervious.

28. A device as defined in claim 20, wherein the liquid impervious container permits the uninterrupted transmission of sound waves therethrough, and is formed of a flexible material.

29. A device as defined in claim 20, wherein a second locating indicia is located on a second side of the first support member, and said second locating indicia is substantially radiopaque when viewed on a radiograph.

30. A device as defined in claim 29, wherein the wherein the second locating indicia is defined by a low density material forming grooves in the first support member.

31. A device as defined in claim 29, wherein the second locating indicia is at least one of a line, a plurality of intersecting lines, a two-dimensional Cartesian plane, a grid pattern, a circle, a plurality of substantially concentric circles, and a combination of any of the foregoing and is substantially in registration with the first locating indicia.

32. A device as defined in claim 31, wherein the low density material further includes alpha-numeric designations formed in the low density material.

33. A device as defined in claim 32, wherein the low density material is substantially plastic.

34. A device of claim 20, further comprising an indicating member that is at least partially radiopaque, and is movably mounted to the first support member, and wherein the indicating member is capable of being disposed to at least two positions indicative of at least a first radiographic image of a tissue specimen, and a second radiographic image of a tissue specimen.

35. A device as defined in claim 20, wherein the second support member is made of a substantially flexible but substantially shape-restoring material, and is formed of at least one of a foam, a rubber, and a plastic.

36. A device as defined in claim 35, wherein the second support member is a low density material so as to be substantially radiolucent.

37. A device as defined in claim 20, wherein the second support member is integral with the first support member.

38. A device as defined in claim 20, wherein the second support member is substantially the same length as the first support member.

39. A device for storing a tissue specimen comprising:
first means defining a sealable, substantially fluid-tight chamber;
second means receivable within the fluid-tight chamber of the first means for supporting a tissue specimen; and
third means for grasping and manipulating from a position external to the fluid-tight chamber a tissue specimen that is located on the second means and sealed within the fluid-tight chamber of the first means.

40. A device as defined in claim 39, wherein the first means is a plastic bag.

41. A device as defined in claim 39, wherein the second means is a tray.

42. A device as defined in claim 39, wherein the third means is a flexible portion of a container that is manually engageable and movable laterally within the fluid-tight chamber for grasping and manipulating a specimen within the chamber.

43. A device for storing a tissue specimen comprising:
a sealable, substantially liquid impervious container;
a support received within the container for supporting a tissue specimen;
an indicating member that is at least partially radiopaque and movably mounted on the support between (i) a first position indicative of a first radiographic image of a tissue specimen located on the support in a first position, and (ii) at least one second position indicative of at least one second radiographic image of the tissue specimen located on the support in at least one second position.

44. A device as defined in claim 43, wherein the second position of the tissue specimen is substantially orthogonal to the first position of the tissue specimen.

45. A device as defined in claim 43, wherein the indicating member is pivotally mounted to the support and is pivotally movable between the first and at least one second positions.

46. A device as defined in claim 43, wherein the indicating member is one of a switch, and a dial movably mounted to said first side of the first supporting member, and wherein said switch or dial has at least two positions and can be disposed to indicate a radiographic image of a tissue specimen located on the first support in a first or second position.

47. A device as defined in claim 43, wherein the indicating member is integrally formed with the first support member.

48. A device as defined in claim 43, wherein the indicating member is integrally formed with a means for movably mounting the indicating member on the first support member.

49. A device as defined in claim 1, wherein the second surface of the first support member contains a radiolucent adhesive to prevent movement of the first support mean within the container.

50. A device as defined in claim 1, further comprising a means for opening a sealed container and resealing the container, and wherein the resealed container is fluid-tight.

51. A method for storing and transporting a tissue specimen comprising the steps of:
providing a device for transporting a tissue specimen, wherein the device includes a container, a flexible portion, a first support member received in the container, a second support member extending from the fist support member, a first locating indicia on a first side of the first supporting member, a second substantially radiopaque locating indicia on a second side of the first support member, and an indicating member movably mounted on the first side of the first support member;
positioning a tissue specimen on the first support member, in a fixed position and then sealing the tissue specimen within the container;
exposing the tissue specimen to x-ray radiation while the specimen is fixed to the first support member and sealed in the container with the indicating member set to a first position to generate an x-ray image of the specimen superimposed on the second locating indicia;
manually engaging the flexible portion and, in turn, grasping and rotating the tissue specimen; and
exposing the tissue specimen to x-ray radiation while the specimen is fixed to the tray and sealed in the container with the indicator member set to a second position to generate an x-ray image of the specimen superimposed on the second locating indicia.
